(19) United States
(12) Patent Application Publication
(10) Pub. No.: US 2011/0130725 A1
(43) Pub. Date: Jun. 2, 2011

(54) CATHETER HOLDER FOR FILTERING MEDICAL FLUIDS

(76) Inventor: Héctor de Jesús Vélez-Rivera, Zapopan (MX)

(21) Appl. No.: 12/664,861

(22) PCT Filed: Jun. 11, 2008
(86) PCT No.: PCT/IB2008/001498

§ 371 Date: Jun. 14, 2010

(30) Foreign Application Priority Data
Jun. 15, 2007 (MX) MA/A/2007007310

(51) Int. Cl. A61M 25/16

(52) U.S. Cl. 604/257

(57) ABSTRACT

Disclosed herein is a catheter holder for filtering medical fluids, comprising a housing with a filter compartment where a filtering sheet is located. Fluid is distributed before passing through the filtering sheet. The catheter holder also has a connector where a medical fluid management device is received. There also exists a connecting chamber where a catheter is retained. The connector, filter compartment and the catheter connecting chamber are in fluid communication. The catheter holder includes an actuator that has “open” and “closed” positions. The actuator is connected to a compression member, which contacts a setscrew. Each time the actuator is moved towards the closed position, the compression member deforms the setscrew, thereby retaining the catheter. The device is preferably used in epidural or spinal anesthesia procedures.

### FIELD OF THE INVENTION

This invention is related to the techniques used in the design of apparatus and equipments for medical purposes, and more specifically, it is related to a catheter holder to filter medical fluids such as medicines or anesthetics that are used in spinal and epidural anesthesia procedures, as well as in other surgical procedures where catheters are used.

### BACKGROUND OF THE INVENTION

Catheters are hollow tubes that can be made of different biocompatible materials and that are widely used in blood transfusions, as well as in the administration of medicines, anesthetics, solutions and other medical fluids. A special use of catheters is found in the spinal and epidural anesthesia, specifically, they are inserted in a patient's backbone with the aid of a Tuohy needle. Often, catheters remain there for some days.

Of course, in the proximal end of a catheter (the closest end to the surgeon) a medical fluid management device must be connected, such as a syringe or an infuser. On the other hand, the distal end of the catheter (the farthest end to the surgeon) is inserted in the patient's body.

There are different apparatus in the prior art known as “catheter adapters”, “catheter connectors” or “catheter holders”, which have been designed to make possible a fluid communication between the fluid management device and the catheter. It must be noted that catheter adaptors must have a hermetic seal to prevent fluid leakages. Besides, the catheter adapters must be easy to assemble in emergency situations, and they must be constructed in order to retain the fluid management device, as well as to keep the catheter in a secure position. If the catheter or the fluid management device is inadvertently separated from the adapter due to a poor connection, the consequences for the surgical procedure the surgeon is performing may turn out to be dangerous.

A catheter adapter that is widely used in the prior art is the one known as “Tuohy-Borst”, which consists of two threaded members, one of them having a female Luer connector to receive a syringe, while the second member is a hollow member wherein an enlarged, compressible “O” ring is housed. In order to assemble the adapter, first, the catheter is inserted in the second member and in the “O” ring, then, as both members are threaded rotating at least once, the “O” ring becomes lengthwise distorted, holding in this way the catheter in its use position.

U.S. Pat. Nos. 5,053,015 and 5,226,898 disclose a catheter adapter that incorporates some improvements regarding the basic configuration of the Tuohy-Borst adapter, one of those improvements is a pair of wings provided in the second threaded member that allows the surgeon to easily handle the adapter. Furthermore, the catheter adapter members are designed such that the accidental unthreading of a member with respect to the other is avoided.

The European Patent No. 0,941,743 B1 discloses another adapter that makes easy the connection of a catheter compared with a Tuohy-Borst adapter, and which comprises a body that has a proximal end, a distal end and a duct therebetween. The duct includes a seat to receive the catheter, and a distal cavity for the catheter seat. Besides, there is a joint inside the cavity to substantially form a hermetic seal around the catheter. The adapter of said European patent further includes a retainer arranged over the proximal end of the body and over the joint with an opening therebetween; this opening is sized to make possible the passageway of the catheter through the duct in order to hold the joint and the catheter seal. The retainer has a plurality of flexible projections inside the opening that are sized and adapted to hold the catheter and to be bent in proximal direction by the catheter placement in the catheter seat. Finally, the adapter has a collar that has a plurality of distal protuberances arranged to hold the flexible projections in the retainer when the collar is in a first position in relation to the body, thus retaining the catheter in the adapter.

The U.S. Pat. No. 6,350,260 describes a catheter coupler which has a very different structure compared with the Tuohy-Borst adapter, the catheter adapter of this document comprises two pivotable jaws that enclose a wavy channel. One of the jaws is provided with a hose in which the catheter can be inserted. When the other jaw is closed, the hose and the catheter become distorted in a wavy shape with no significant change in its cross section; in this way, the catheter is fastened to avoid disconnection. However, during the insertion of the catheter in the hose, a slight force must be applied in order to lead the catheter proximal end to the hose proximal end for a secure connection; such movement is considered a drawback for the catheter coupler since it consumes time.

On the other hand, in order to separate the catheter from the hose compressed by jaws, a user has to operate the catheter adapter by its ends and to apply, with the aid of its fingers, an upward force in the bottom and in the center of the lower jaw, this force causes both jaws disengagement, and then the catheter can be removed from the hose.

At this point, is very important to say that, in the epidural anesthesia procedures, filtering drugs and anesthetics is required to retain any contaminant or microorganism that may be in the fluid that is going to be supplied. On the subject, none of the prior art aforementioned adapters include a filter to fulfill this requirement. In the past and nowadays, a common practice to carry on this purpose has been to place a filter before the catheter adapter. As it can be brought forward, the time the surgeon took to connect the filter between the fluid management device and the adapter is increased; time is a decisive factor in epidural anesthesia. Another problem in this practice is that filters should be sterilized using some special equipment, and once the filter and the catheter adapter are used, they have to be discarded.

A solution to this problem regarding the use of a filter and a catheter adapter is disclosed in the Mexican utility models No. 1188 and 1267, in these documents, a catheter adapter is provided with a filter located inside the adapter. Nevertheless, the way to retain the catheter and the syringe in the catheter adapter is that used in the Tuohy-Borst-type adapter; that is, several turns among the threaded members are required to secure the catheter. Besides, due to the inclusion of a filter in the adapter body, the adapter shape is not ergonomic to achieve an easy management and use of the adapter; more specifically, the adapters of said utility models have a disc portion where the filter is housed.

From the previous, it can be noted that there is a need to filter the drugs and anesthetics that are supplied to the patient's body through a catheter. In the prior art, there is a lack of catheter adapters with an incorporated filter and with a structural and ergonomic design for a fast connection of the catheter.

### SUMMARY OF THE INVENTION

A solution has been developed in order to avoid the problems of the prior art catheter adapters, connectors and holders which are used in epidural and spinal anesthesia procedures; the developed solution is particularly related with a catheter holder to filter medical fluids that comprises: a housing that has a proximal end and a distal end; a connector that receives a fluid management device and that has a medical fluid; the connector is formed in the proximal end of the housing. Furthermore, there is a filter compartment which is formed inside said housing and which is in fluid communication with the connector. Of course, there are media to filter the fluid, which are located inside the filter compartment. On the matter, the fluid is distributed in the filter compartment before passing through the filtering media.

As a part of the holder of the present invention, there is a catheter connecting chamber which is formed inside the housing and which is in fluid communication with the filter compartment. Besides, the holder has a set screw that has a central passageway and which is inserted in the catheter connecting chamber. Another element of the catheter holder of the present invention is a compression member which is in contact with the set screw; the compression member moves inside the catheter connecting chamber. In the present invention, an end portion of the catheter is received along the compression member and the central passageway of the set screw.

Finally, the catheter holder to filter medical fluids of the present invention comprises an actuator connected to the compression member and which is moveably mounted in the housing. The actuator has an “open” position and a “closed” position, such than when the actuator is moved to the “closed” position, the compression member moves inside the housing and lengthwise distorts said set screw, thereby retaining the end portion of the catheter along the central passageway of said set screw. On the other hand, when the actuator is moved to the “open” position, the compression member moves outside the housing and decompresses the set screw, through which the end portion of the catheter may be inserted in or removed from the set screw and the compression member.

As it was mentioned, the actuator is movably mounted in the housing so that when it moves to a predetermined direction or to another, the “closed” position or the “open” position is reached, which allows securing and removing the catheter. That is, on moving, the actuator should move, at the same time, the compression member that distorts the set screw. The movement of the actuator can be sliding, pivotable, rotating up to 180° or swinging in the housing and it can be provided as a button or a wing with such movement.

In a first preferred embodiment of the present invention, the actuator can move in a sliding manner and it comprises a pair of flexible wings, each of which slides inside the housing and extends from the compression member to a lateral side of the housing; each wing has a thrust projection integrally attached thereof, the thrust projection protrudes from the lateral side of the housing in order to be located and moved by the user's finger.

In each wing, a locking element is provided and it runs in a sliding channel formed inside the housing. The locking element engages in a cooperating cavity located in each end of the sliding channel when the actuator is laterally pushed and moved to reach the “open” or “closed” position.

In a second preferred embodiment of the present invention, the actuator is rotating with a controlled and limited movement of 180°, when the actuator rotates such angle in one sense or in another, it makes the catheter be retained or removed, that is, the actuator reaches its “open” or “closed” position. In this second embodiment, the actuator comprises a rotating wing integrally attached to the compression member, which is provided with a male thread to rotate inside the catheter retaining chamber, which is provided with a female thread. When the actuator rotates 180°, the compression member also rotates and compresses the set screw and in this way the catheter is secured. To keep the “closed” position of the actuator, the housing includes a retainer provided in the distal port, the actuator being fixed under the retainer. Likewise, to limit the movement of the actuator, the housing includes a horizontal platform that protrudes from the distal end thereof. The platform has such a size that only a portion of the actuator laterally protrudes from it to take it.

As it could be noted from the aforementioned, it is an object of the present invention to provide a catheter holder to filter medical fluids, preferably, drugs and anesthetics.

Another object of the present invention is to provide a catheter holder to filter medical fluids, which allows surgeons to connect a catheter and a fluid management device to the catheter holder in a short time.

### BRIEF DESCRIPTION OF THE DRAWINGS

The novel aspects that are considered characteristic of the present invention will be particularly set in the attached set of claims. However, the invention itself, either for the structural organization of its elements or for its operation method, along with other objects and advantages thereof, will be understood in the following description of a preferred embodiment thereof, reading it regarding the attached drawings, wherein:

FIG. 1 is a perspective view from the proximal end of a catheter holder to filter medical fluids built according to a first preferred embodiment of the present invention.

FIG. 2 is a side view of the catheter holder o filter medical fluids shown in FIG. 1.

FIG. 3 is a top plan view of the catheter holder to filter medical fluids shown in FIG. 1.

FIG. 4 is an exploded view of the catheter holder to filter medical fluids shown in FIG. 1.

FIG. 5 is a top plan view of the lower half of the housing that pertains to the catheter holder shown in FIG. 4.

FIG. 6 is a cross section view taken along the A-A′ line of the catheter holder of FIG. 1, showing the catheter holder in its “open” position.

FIG. 7 is a cross section view taken along the A-A′ line of the catheter holder of FIG. 1, showing the catheter holder in its “closed” position.

FIG. 8 is an upper perspective view of a second embodiment of the catheter holder to filter medical fluids of the present invention.

FIG. 9 is an exploded view of the catheter holder to filter medical fluids shown in FIG. 8.

FIG. 10 is a cross section view taken along B-B′ line of the catheter holder of FIG. 8, showing the catheter holder in its “open” position.

FIG. 11 is a cross section view taken along B-B′ line of the catheter holder of FIG. 8, showing the catheter holder in its “closed” position.

### DETAILED DESCRIPTION OF THE INVENTION

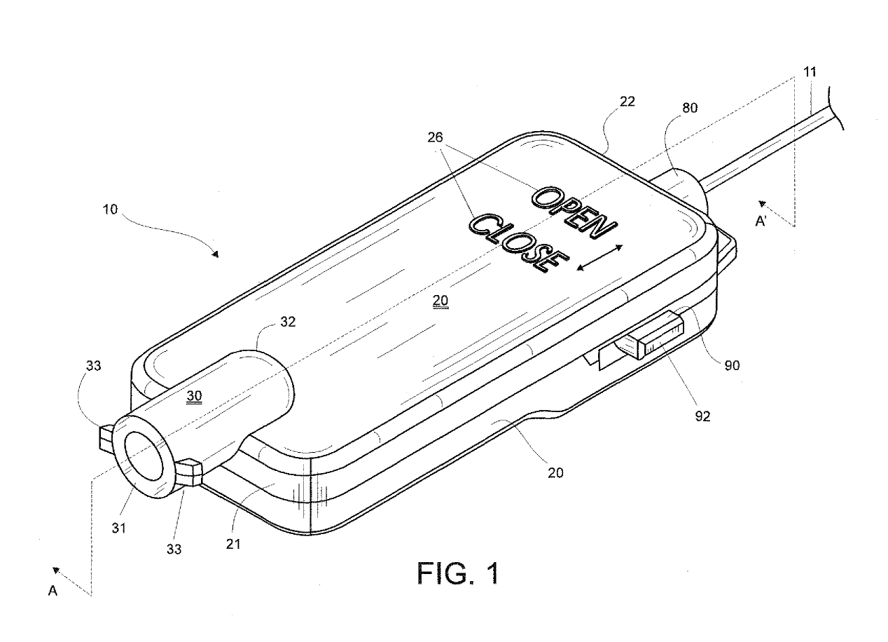

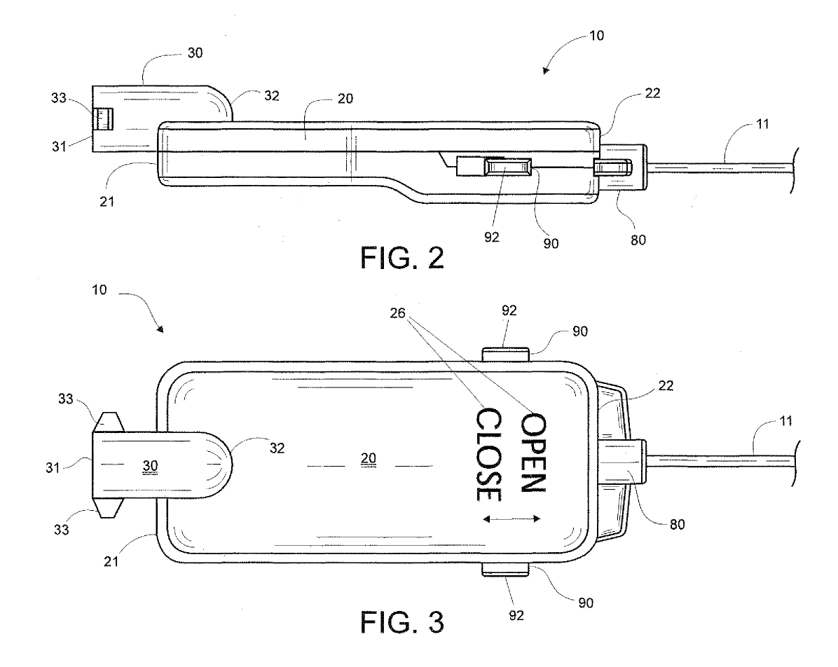

In FIGS. 1 to 3 of the attached drawings, a catheter holder can be observed identified with number 10 which is used to filter medical fluids, such as anesthetics or drugs. The catheter holder 10 is built according to the principles of a first preferred embodiment of the present invention, which must be considered as illustrative rather than restrictive thereof. The catheter holder 10 comprises a housing 20 that has a proximal end 21 and a distal end 22. As previously mentioned, the distal end corresponds to the direction towards the patient where the catheter 11 is inserted, and the proximal end is that closer to the surgeon.

The catheter holder of the present invention has a connector to receive a medical fluid management device, preferably a syringe or a medical infuser (not shown) and which contains a medical fluid that must be filtered. In the first preferred embodiment shown in FIGS. 1 to 3, the connector is provided in the shape of a tube 30 integrally formed at the proximal end 21 of the housing 20. The tube 30 has a proximal end 31 and a distal end 32; of which the proximal end 31 has a luer female connector 33. On this regard, the luer connectors are widely used in medical field and there is no need to be described in detail in the present descriptive text. On the other hand, the distal end 32 of the tube 30 is inserted in the housing 20 through an upper surface thereof.

In FIGS. 1 to 3, it can also be observed a compression member 80 and an actuator 90 that are connected to each other, the interaction of these elements with a set screw that is inside the housing 20 allows the catheter 11 to be retained in the catheter holder 10 as it will be explained later. Even more, the housing 20 has tactile or visual marks 26 that indicate if the actuator 90 is in “open” or “closed” position. The actuator 90 is moved and pushed by the thrust protrusions 92.

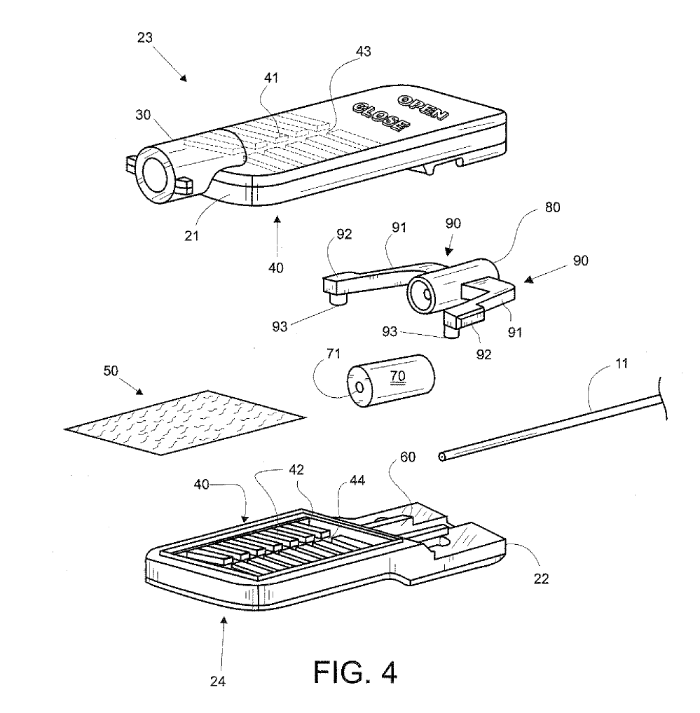

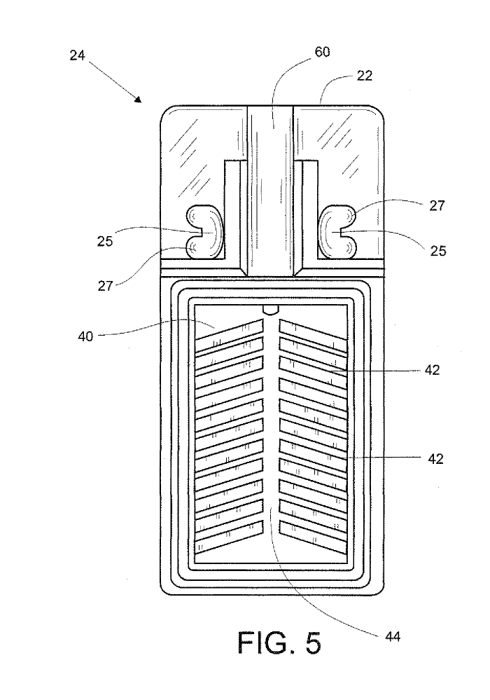

Now, reference is made to FIGS. 4 and 5, in order to explain that the housing is made up an upper half 23 and a lower half 24; when both halves are assembled, a filter compartment 40 is formed inside of the housing. This filter compartment 40 is in fluid communication with the tube 30 such that the fluid is received first in the tube 30 and then it is directed to the filter compartment 40, which has a first plurality of distribution channels 41 that run through the upper part of said filter compartment 40, the channels 41 are represented by broken lines in FIG. 4, additionally a second plurality of channels 42 is included, which runs along the bottom of the filter compartment 40.

It can be mentioned that, for this embodiment, the filtering media are a filtering sheet 50 located between the first and second plurality of channels 41 and 42. The first plurality of channels 41 homogeneously distributes the medical fluid before passing through the filtering sheet 50, while the second plurality of channels 42 receives the fluid that has passed through the filtering sheet and it is led to a catheter connecting chamber 60, which will be described subsequently. Channels of the first plurality of channels 41 are in fluid communication with each other by a first central channel 43 (represented by broken lines) which runs along the upper part of the filter compartment 40. On the other hand, channels of the second plurality of channels 42 are in fluid communication with each other by a second central channel 44 that runs along the bottom of the filter compartment 40.

Channels of the first and second plurality of channels 41 and 42 are arranged in the filter compartment 40 in a “V” arrangement, wherein channels of the first and second plurality of channels 41 and 42 are parallel to each other. On this matter, it is important to mention that the gap distance between the pair of adjacent channels of the first plurality of channels 41 is larger than the existing between a pair of adjacent channels of the second plurality of channels 42. This dimensional difference between channels 41 and 42 allows the medical fluid to pass through the filtering sheet 50, since a pressure difference is generated between channels 41 and 42. Within the present invention, it is also possible to use other kind of arrangement for channels 41 and 42, such as a diagonal channel arrangement, a longitudinal or cross channel arrangement, circular channels or any other kind of arrangement that makes possible the distribution of fluid before passing through the filtering media and collecting it once it has passed through it.

As it was said, the housing is made up of an upper half 23 and a lower half 24 that are joined such that the filtering sheet 50 is fixed between both halves 23 and 24. The housing is preferably made of a medical grade plastic. Even more, for a simple management of the catheter holder 10, the housing 20 has an elongated parallelepipedon shape that could be taken laterally between the forefinger and the thumb. Nevertheless, other forms for the filtering media as well as for the housing 20 are possible, trying the housing to have an ergonomic shape in order to be easily grabbed.

Each time the catheter holder 10 is used, the residual volume of the medical fluid that is retained by the filtering media is lower than about 10 ml. The residual volume is a key factor for the catheter holder use and it represents the remaining amount of fluid in the filtering media once the catheter holder 10 has been used. The catheter holder 10 of the present invention is suitable for filtering fluids as drugs, anesthetics and solutions used in epidural and spinal anesthetics procedures, but, according to the invention, there is no restriction in the fluid to be filtered.

To achieve the filtration object, filtering media have a pore diameter lower than about 30 microns, for the embodiment being described, the filtering sheet 50 can be made up of polyethersulfone (PES), polytetrafluoroethylene/Teflon® or the like that retains impurities and microorganisms.

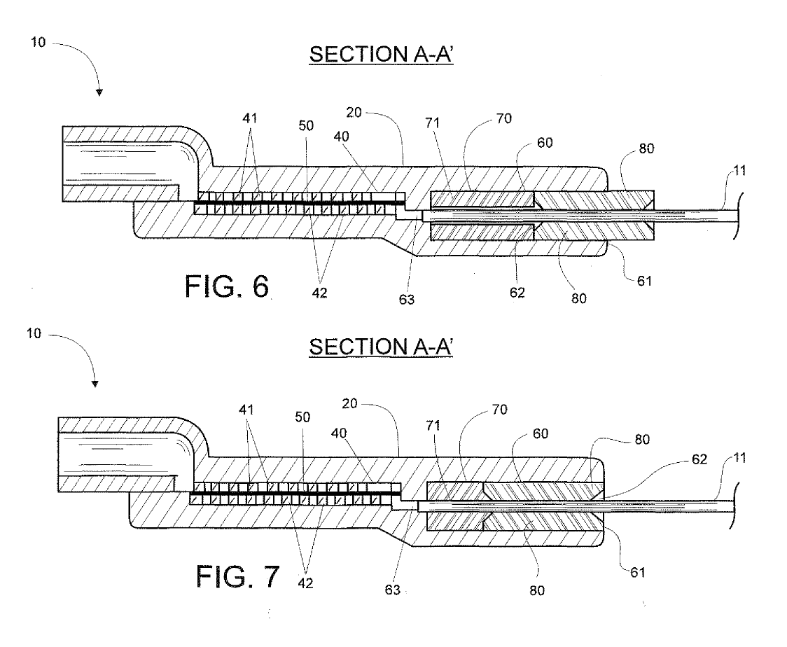

Now, reference to FIGS. 4 and 5 is made along with FIGS. 6 and 7 to describe the way the catheter 11 is retained by the catheter holder 10 of the present invention. For this purpose, the catheter connecting chamber 60 is in fluid communication with the filter compartment 40, more specifically, the catheter connecting chamber 60 is formed inside the housing and it is located near the distal end 22 thereof.

Inside the catheter connecting chamber 60, a set screw 70 is inserted, which preferably is elastic, tubular shape and it has a central passageway 71, the set screw 70 is in contact with the compression member 80, which is hollow and can be slid inside and outside of the catheter connecting chamber 60. An end portion of the catheter 11 is received along the compression member 80 and the central passageway 71 of the set screw 70 (see FIGS. 6 and 7).

Regarding the compression member 80, it is connected to the actuator 90, which at the same time is moveably mounted in the housing 20. The actuator 90 has an “open” position and a “closed” position, in such a way that when the actuator 90 is moved to the “closed” position, the compression member 80 slides inside the housing and lengthwise distorts said set screw 70, thereby retaining the end portion of the catheter 11 along the central passageway 71 of the setscrew 70 as it is particularly shown in FIG. 7. On the contrary, when the actuator 90 is moved to the “open” position, the compression member 80 is slid outside of the catheter connecting chamber 60, in this way decompressing the setscrew 70, thereby the end portion of the catheter 11 can be removed from or inserted in the set screw 70 and the compression member 80 (see FIG. 6).

The actuator 90 can also be moveably mounted in the housing 20 in a rotating or swinging way, etc., provided that the actuator 90 is in contact with the compression member 80 and causes the latter to distort the set screw 70 lengthwise. The compression member 80 has a shape such that allows longitudinal distortion of the set screw. In the embodiment being described, the compression member 80 has a hollowed cylinder shape in order to allow the end portion of the catheter 11 pass and be inserted in the set screw 70.

In order to disclose some additional features of the actuator 90 employed in the embodiment being disclosed, special reference is made to FIGS. 4 and 5, from which it can be observed that the actuator 90 comprises one pair of flexible wings 91, each one sliding inside the housing and extending from the compression member 80 to a lateral side of the housing 20. Each wing 91 has a thrust projection 92 integrally attached thereto, said thrust projection 92 protruding from a lateral side of the housing 20 to be easily located and moved by a finger of the user. This feature can also be seen in FIG. 3, which shows a top plan view of the catheter holder 10.

Referring again to FIGS. 4 and 5, it can be noticed that the actuator 90 additionally has a locking element 93 provided at each wing 91 and running inside the sliding channel 25 formed inside the housing, more specifically, the sliding channel 25 is formed at the lower half 24 of the housing 20 and is laterally separated from said catheter connecting chamber 60 (see FIG. 5 for further detail). The locking element 93 is coupled inside a co-operating cavity 27 located at each end of the sliding channel 25 when the actuator 90 is laterally pushed and moved in order to reach the “open” or “closed” position. The locking element 93 protrudes underneath its respective wing 91, whereas the sliding channel preferably has a “C” shaped pathway. In FIG. 5 it can also be seen the filter compartment 40, the second plurality of channels 42 and the second central longitudinal channel 44, the function of which at the holder 10 has previously been described.

On the other hand, from FIGS. 6 and 7 it can also be mentioned that the catheter connecting chamber 60 includes an entrance 61 where the compression member 80 slides inside and outside of the catheter connecting chamber 60; one middle portion 62, in which the set screw 70 is inserted and distorted by the compression member 80, the middle portion 62 is located next to the entrance 61. A seat 63 is located next to the middle portion 62 and has a diameter that matches in size the outer diameter of the catheter, such that the seat 63 is fully taken up by the proximal end of the catheter 11 so as to prevent leakage of the medical fluid coming from the filter compartment 40. FIGS. 6 and 7 also show the filtering sheet 50 between the first and second plurality of channels 41 and 42.

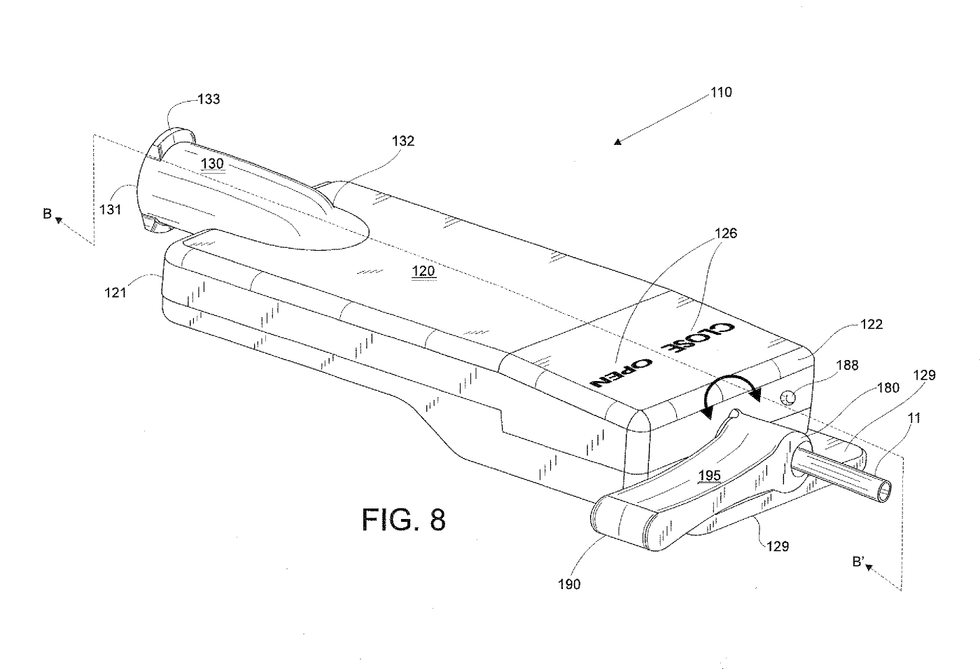

Reference is now made to FIG. 8, which shows a second preferred embodiment of the present invention, which is identified with number 110. For this second embodiment, the common elements shared with the first embodiment of the previous figures will have the same numerals but with the “1” numerical prefix added thereto. This second embodiment is characterized in that the actuator 190 rotates with a controlled movement of 180° or of a half turn.

The catheter holder 110 of FIG. 8 comprises a housing 120 that has a proximal end 121 and a distal end 122. In the proximal end 121 there is located a connector for medical fluids, essentially comprising a tube 130 integrally formed at the proximal end 121 of the housing 120. The tube 130 in turn has a proximal end 131 and a distal end 132, of which the proximal end 131 has a female luer connector 133. On the other hand, the distal end 132 of the tube 130 is inserted inside the housing 120 through the upper surface thereof.

In FIG. 8 it is additionally shown a compression member 180 and the actuator 190 integrally attached to said compression member; interaction of these elements with a set screw that is inside the housing 120 allows the catheter 11 to be retained in the catheter holder 110, as explained below.

The housing 120 has tactile or visual markings 126 that indicate whether the actuator 190 is in an “open” or “closed” position (open-closed). The actuator 190 essentially comprises a rotating enlarged wing 195 with a restricted and controlled movement of 180° or of a half turn. Furthermore, the housing 120 has at the distal end thereof a horizontal platform 129 that restricts the rotating movement of the enlarged wing 195 to prevent it from rotating beyond the allowed angle; additionally, it has a retainer in the shape of a protuberance 188 to secure the enlarged wing 195 when the actuator is in the closed position.

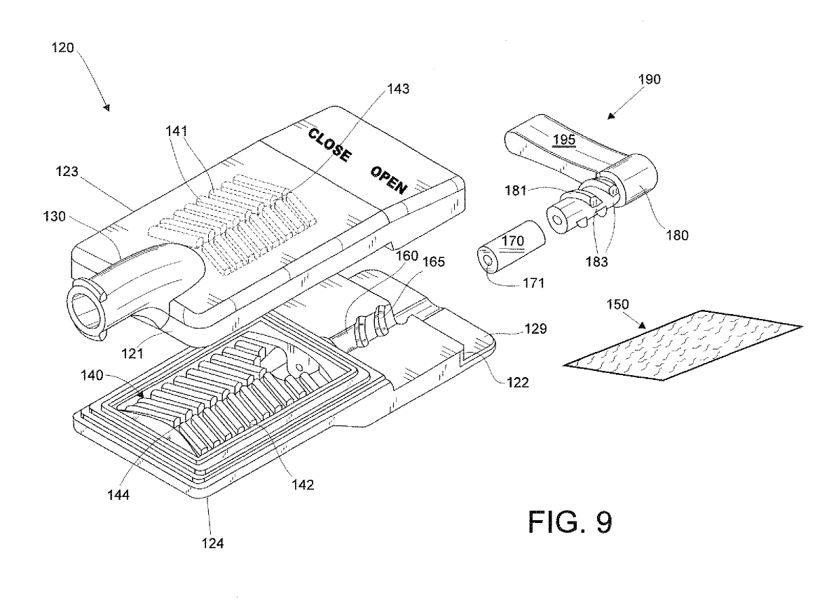

At this moment, it is convenient to refer to FIG. 9 in order to explain that the housing 120 is formed by an upper half 123 and a lower half 124; when both halves are assembled together, a filter compartment 140 is formed inside the housing 120. This filter compartment 140 is in fluid communication with the tube 130, such that the fluid is received firstly in the tube 130 and subsequently directed to the filter compartment 140, which has a first plurality of distribution channels 141 running through the upper part of said filter compartment 140. The channels 141 are represented by broken lines in FIG. 9; additionally, a second plurality of channels 142 is included that run along the filter compartment 140 bottom.

It can be said that, for this second embodiment, the filtering media again comprise a filtering sheet 150 located between the first and second plurality of channels 141 and 142. The first plurality of channels 141 homogeneously distributes the medical fluid before going through the filtering sheet 150, whereas the second plurality of channels 142 receives the fluid that has passed through the filtering sheet and leads it towards a catheter connecting chamber 160. The channels of the first plurality of channels 141 are in fluid communication with each other by means of a first central channel 143 (shown as broken lines) running through upper part of the filter compartment 140. On the other hand, the channels of the second plurality of channels 142 are in fluid communication with each other by means of a second central channel 144 running along the bottom of the filter compartment 140.

Channels of the first and second plurality of channels 141 and 142 are arranged in the filter compartment 140 in a “V” arrangement, wherein the channels of the first and second plurality of channels 141 and 142 are parallel to each other. The gap distance between a pair of adjacent channels of the first plurality of channels 141 is larger than that existing between a pair of adjacent channels of the second plurality of channels 142. This dimensional difference between channels 141 and 142 allows the medical fluid to pass through the filtering sheet 150, since a pressure difference is created between channels 141 and 142.

As previously mentioned, the housing 120 is formed by one upper half 123 and one lower half 124 attached in such a way that the filtering sheet 150 is secured between both halves 123 and 124, Each time the catheter holder 110 is used, the residual volume of medical fluid retained by the filtering media is less than around 10 ml. In this second embodiment, the filtering media are identical to those described for the first embodiment, i.e. a pore diameter of under 30 microns approximately. Thus, the filtering sheet 150 can be made of polyethersulphone (PES), Teflon® polytetrafluoroethylene or other similar filtering material that retains impurities and microorganisms.

Now, reference is made to FIGS. 9 to 11 to describe the manner in which the catheter 11 is retained by the catheter holder 110 of the second embodiment. For this purpose, the catheter connecting chamber 160 is in fluid communication with the filter compartment 140 and is provided with a female thread 165; more specifically, the catheter connecting chamber 160 is formed inside the housing 120 and is located next to the distal end 122 thereof.

Inserted inside the catheter connecting chamber 160 there is a set screw 170 which is preferably elastic, with a tubular shape and a central passage 171; the set screw 170 contacts the compression member 180, which is hollow and can be rotated 180° maximum inside the catheter connecting chamber 160. in this way, an end portion of the catheter 11 is received along the compression member 180 and the central passage 171 of the set screw 170.

The compression member 180 has a discontinuous male thread 181, i.e. the male thread is interrupted at the flat zones 183 every 180°, this is with the purpose of facilitating the fast retaining and releasing of the catheter, provided that when the enlarged wing 195 of the actuator is taken to the “closed” position (FIG. 13); the actuator should have axial movement freedom in order to surround and remain underneath the retainer 188, which is a cylindrical protuberance, the retainer 188 maintains the actuator 190 in this “closed” position. On the contrary in order to reach the “open” position, the actuator 190 is simply rotated 180° in the opposite direction. In addition to the retainer 188, the rotating movement of the actuator 190 is limited by a horizontal platform 129 extending from the distal end of the housing; a portion of the enlarged wing 195 laterally protrudes from the platform 129 in order to be manipulated, the platform avoiding the movement of the wing to be greater than 180°.

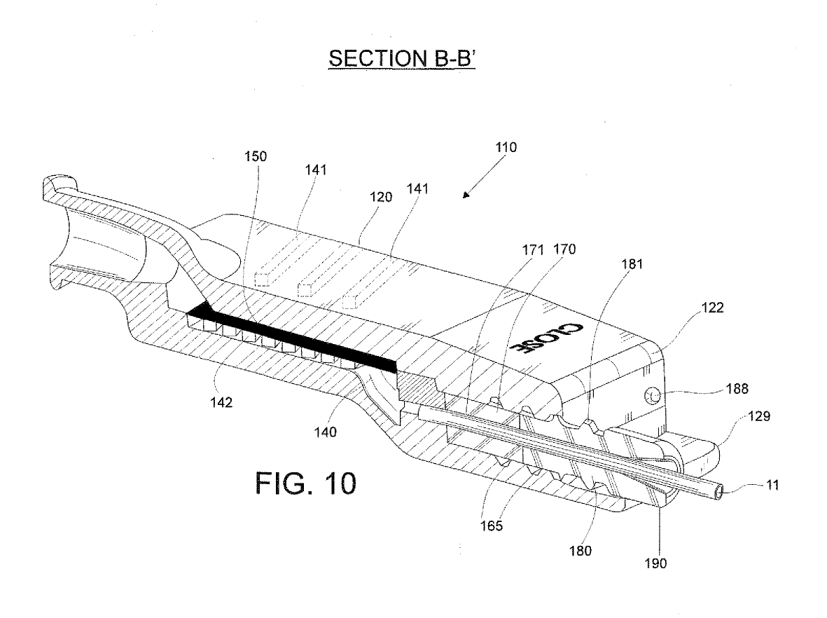

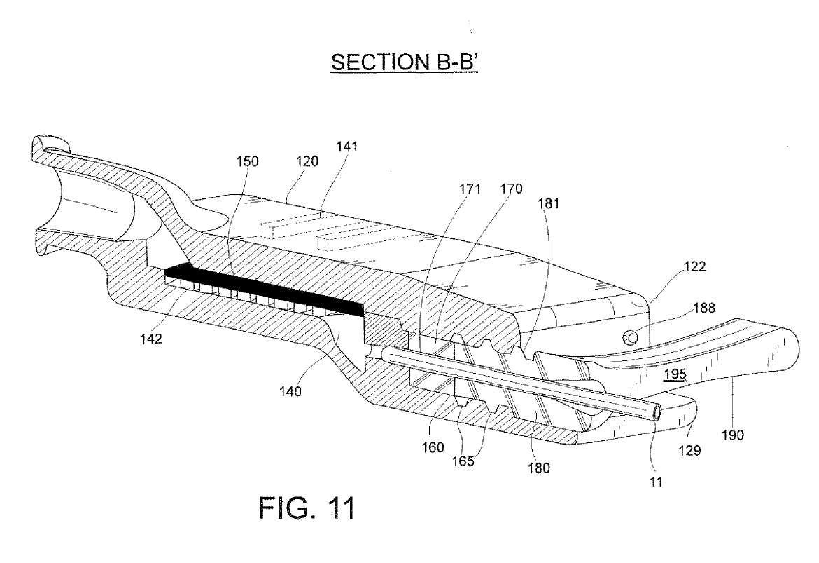

On the other hand, from FIGS. 10 and 11 it can also be mentioned that the catheter connecting chamber 160 includes an entrance 161 where the compression member 180 slides inside and outside of the catheter connecting chamber 160; one middle portion 162, in which the set screw 170 is inserted and distorted by the compression element 180, the middle portion 162 is located next to the entrance 61. A seat 163 is located next to the middle portion 162 and has a diameter that matches in size the outer diameter of the catheter, such that the seat 163 is fully taken up by the proximal end of the catheter 11 so as to prevent any leakage of the medical fluid coming from the filter compartment 40. FIGS. 6 and 7 also show the filtering sheet 150 between the first and second plurality of channels 141 and 142.

According to the embodiments previously disclosed and shown in the attached drawings, it can be seen that the catheter holder used for filtering medical fluids has been designed for an easy handling thereof. However, both the fluid management device and the catheter are securely and rapidly connected to the catheter holder. Hence, it will be obvious for an expert in the art that the embodiments previously disclosed are only illustrative and do not restrict the invention. Several modifications can be made thereon, such as the shape of the housing, the type of connector of the fluid management device, the manner in which the actuator is moveably assembled in the housing, among other, without departing from the scope and essential features of the invention.

Even though in the previous disclosure a preferred embodiment of the present invention has been shown and described, the fact that numerous modifications thereto are possible must be highlighted. Therefore, the present invention should not be restricted by the state of the art and by the appended claims.

1. A catheter holder for filtering medical fluids, characterized because it comprises:
a) a housing having a proximal end and a distal end;
b) a connector to receive a fluid management device that contains a medical fluid; said connector being formed at the proximal end of said housing;
c) a filter compartment that is formed inside said housing and in fluid communication with said connector;
d) filtering media for the fluid, which are located inside said filter compartment, wherein the fluid is distributed inside said filter compartment prior to passing through said filtering media;
e) a catheter connecting chamber that is formed inside the housing and in fluid communication with said filter compartment;
f) a set screw having a central passageway and that is inserted in said catheter connecting chamber;
g) a compression member in contact with the set screw; said compression member moving inside the catheter connecting chamber; wherein an end portion of the catheter is received along the compression member and at the central passageway of the set screw;
h) an actuator connected to said compression member and moveably mounted in the housing, the actuator having an “open” and “closed” position, such that when the actuator is moved into the “closed” position, the compression member moves inside the housing and lengthwise distorts said set screw, thereby retaining the end portion of the catheter along the central passageway of said set screw; and, when the actuator is moved to the “open” position, the compression member moves outside the housing and decompresses the set screw, through which said catheter portion can be inserted in or removed from the set screw and the compression member.

2. A catheter holder for filtering medical fluids, according to claim 1, further characterized because said connector is a tube which is integrally formed at the proximal end of said housing and has proximal and distal ends; of which, the proximal end has a female luer connector, whereas the distal end is inserted in the housing.

3. A catheter holder for filtering medical fluids, according to claim 1, further characterized because, each time the holder is used, said filtering media retain a residual volume of medical fluid lower than 10 ml.

4. A catheter holder for filtering medical fluids, according to claim 3, further characterized because the medical fluid is a medicament, an anesthetic or a solution.

5. A catheter holder for filtering medical fluids, according to claim 1, further characterized because said filtering media are a filtering sheet.

6. A catheter holder for filtering medical fluids, according to claim 5, further characterized because said filtering sheet is made of polyethersulphone (PES) or polytetrafluoroethylene Teflon®.

7. A catheter holder for filtering medical fluids, according to claim 1, further characterized because said filtering media has a pore size lower than 30 microns.

8. A catheter holder for filtering medical fluids, according to claim 1, further characterized because said filter compartment comprises:
a first plurality of distribution channels in fluid communication with each other to homogeneously distribute the fluid before passing through said filtering media; and,
A second plurality of distribution channels in fluid communication with each other to receive the fluid that has passed through the filtering media and guide the same towards said catheter connecting chamber;
wherein said filtering media are located between the first and second plurality of channels.

9. A catheter holder for filtering medical fluids of claim 8, wherein the distribution channels of the first and second plurality of channels are laid in an arrangement selected from the group comprising a longitudinal channel arrangement, cross channels, “V” channels, diagonal channels and circular channels.

10. A catheter holder for filtering medical fluids of claim 1, wherein the compression member is shaped such that it allows the longitudinal deformation of the setscrew.

11. A catheter holder for filtering medical fluids of claim 1, wherein said actuator is slidably, pivotably, swingedly or rotatingly mounted in the housing, such that when moving in a predetermined of a different direction, the “closed” or “open” position can be reached that allows the securing or removal of the catheter.

12. A catheter holder for filtering medical fluids of claim 11, wherein said actuator comprises:
a pair of flexible vanes, each one sliding inside the housing and extending from the compression member towards a lateral side of the housing; each vane having a thrust protrusion integrally attached thereto, the thrust protrusion protruding from the lateral side of the housing to be located and moved by the finger of a user; and
a locking element provided in each vane running in a sliding channel shaped inside the housing; the locking element being coupled inside a co-operating cavity located at each end of the sliding channel when the actuator is laterally pushed and moved to reach the “open” or “closed” position.

13. A catheter holder for filtering medical fluids of claim 12, wherein the locking element protrudes underneath the respective vane.

14. A catheter holder for filtering medical fluids of claim 12, wherein the sliding channel has a “C”-shaped pathway.

15. A catheter holder for filtering medical fluids of claim 11, wherein the actuator has a rotating movement of 180° and is integrally attached to the compression member, which is provided with a male thread and the catheter connecting chamber is provided with a female thread; when the actuator is rotated towards the “closed” position, the compression member rotates and compresses the setscrew and thereby secures the catheter, the housing further comprising a retainer provided at the distal port such that the actuator maintains its “closed” position.

16. A catheter holder for filtering medical fluids of claim 15, wherein the housing includes a horizontal platform provided at the distal end thereof, the platform having a size such that a portion of the actuator protrudes therefrom in order to handle the same.

17. A catheter holder for filtering medical fluids of claim 15, wherein the male thread of the compression member is interrupted every 180° to allow the actuator axial movement freedom in order to surround and remain underneath the retainer when moved towards the actuator's “closed” position.

18. A catheter holder for filtering medical fluids of claim 15, wherein the actuator has an enlarged vane shape.

19. A catheter holder for filtering medical fluids of claim 1, wherein said catheter connecting chamber comprises:
an entrance wherein the compression member slides in and out of said housing,
a middle portion, in which the setscrew is embedded and becomes deformed by the compression member; the middle portion being located next to the entrance; and
a seat located next to the middle portion and having a diameter that matches in size the outer diameter of the catheter, such that the seat is fully taken up by the proximal end of the catheter.

20. A catheter holder for medical fluids of claim 1, wherein said housing is formed by a lower half and an upper half that are attached in a way that said filtering media are fixed between both halves.

21. A catheter holder for filtering medical fluids of claim 1, wherein said housing is made of a medical grade plastic.

22. A catheter holder for filtering medical fluids of claim 1, wherein said housing has an enlarged parallelepiped shape that can be grabbed sideways with the thumb and index finger of a user.

23. A catheter holder for filtering medical fluids of claim 1, wherein the actuator has tactile and/or visual markings which indicate whether the actuator is in the “closed” or “open” position.
